(19) United States
(12) Patent Application Publication
(10) Pub. No.: US 2018/0055961 A1
(43) Pub. Date: Mar. 1, 2018

(54) APPARATUS AND METHOD FOR SANITIZING WEARABLE EQUIPMENT

(71) Applicant: CLEANQUIP SYSTEMS INC, London (CA)

(72) Inventor: Paul NOAD, London (CA)

(73) Assignee: CLEANQUIP SYSTEMS INC, London (CA)

(21) Appl. No.: 15/688,612

(22) Filed: Aug. 28, 2017

Related U.S. Application Data
(60) Provisional application No. 62/379,494, filed on Aug. 25, 2016.

(51) Int. Cl. A61L 2/10; A61L 2/24

(52) U.S. Cl. CPC A61L 2/10; A61L 2/26; A61L 2/24

(57) ABSTRACT

There is disclosed a system and method for sanitizing wearable equipment. In an embodiment, the system comprises a power source; a plurality of sanitizing modules arranged on a platform; and a control module with a user interface for controlling the plurality of sanitizing modules. In a preferred embodiment, the platform is a mobile trailer having a power generator. The plurality of sanitizing modules on board the platform may include one or more of an ozonator module, a pro-biotic spray module and a UV light module. The system may further comprise a dehumidifier module and a thermostat module for controlling one or more of a heater and an air conditioner.

### RELATED CASES

The present application claims the benefit of U.S. Provisional Application No. 62/379,494, filed on Aug. 25, 2016.

### FIELD OF THE INVENTION

The present invention relates generally to sanitizing, and more particularly to an apparatus and method for sanitizing wearable equipment.

### BACKGROUND

Wearable equipment is used in various occupations and fields of endeavor, including sports equipment worn by football, baseball and hockey players, safety equipment worn by workers in various occupations including firefighters, police, the military, and work-site safety gear worn by workers onsite.

When worn during activities involving physical exertion, wearable equipment can quickly become soaked with perspiration and soiled, leading to stale equipment that is unpleasant to wear. However, sanitizing such wearable equipment can be inconvenient and challenging, as the wearable equipment can be very bulky to transport, and ill-suited to more conventional methods such as laundry or dry cleaning. Sanitizing wearable equipment for an entire sports team, or an entire department of firefighters or police at a station potentially becomes even more inconvenient and cumbersome, and therefore the stale equipment is often left for lengthy periods, possibly leading to accumulation of harmful bacteria and microorganisms that may lead to infections.

What is needed is an improved system and method for sanitizing wearable equipment which addresses at least some of the limitations in the prior art.

### SUMMARY

The present invention relates to a system and method for sanitizing wearable equipment, and more particularly to a mobile system and method for sanitizing wearable equipment onsite.

In an aspect, there is provided a system for sanitizing wearable equipment, comprising: a plurality of sanitizing modules arranged on a platform within a sealable enclosure; and a control module with a user interface for remotely controlling the plurality of sanitizing modules.

In an embodiment, the platform is mobile.

In another embodiment, the platform is a mobile trailer having a mobile power generator.

In another embodiment, the plurality of sanitizing modules comprises one or more of an ozonator module, a pro-biotic spray module and a UV light module.

In another embodiment, the system further comprises a dehumidifier module and a thermostat module for controlling one or more of a heater and an air conditioner.

In another embodiment, the control is configured to control one or more automated sanitizing programs based on the type of wearable equipment to be sanitized.

In another embodiment, the sealable enclosure is accessible via a sealable access door.

In another embodiment, the sealable access door provides access to a loading area for loading wearable equipment to be sanitized.

In another embodiment, the system further comprises a rack for loading and unloading wearable equipment to be cleaned without having to enter the sealable enclosure.

In another embodiment, the system further comprises a conveyor for loading and unloading wearable equipment to be cleaned without having to enter the sealable enclosure.

In another aspect, there is provided a method of sanitizing wearable equipment, comprising: providing a plurality of sanitizing modules arranged on a platform within a sealable enclosure; and providing a control module with a user interface for remotely controlling the plurality of sanitizing modules.

In an embodiment, the method further comprises sanitizing the wearable equipment by utilizing one or more of an ozonator module, a pro-biotic spray module and a UV light module.

In another embodiment, the method further comprises utilizing a dehumidifier module and a thermostat module for controlling one or more of a heater and an air conditioner.

In another embodiment, the control is configured to control one or more automated sanitizing programs based on the type of wearable equipment to be sanitized.

In another embodiment, the method further comprises providing access to the sealable enclosure via a sealable access door.

In another embodiment, the method further comprises providing a loading area adjacent the sealable access door for loading wearable equipment to be sanitized.

In another embodiment, the method further comprises providing a rack for loading and unloading wearable equipment to be cleaned without having to enter the sealable enclosure.

In this respect, before explaining at least one embodiment of the invention in detail, it is to be understood that the invention is not limited in its applications to the details of construction and to the arrangements of the components set forth in the following description or the examples provided therein, or illustrated in the drawings. The invention is capable of other embodiments and of being practiced and carried out in various ways. Also, it is to be understood that the phraseology and terminology employed herein are for the purpose of description and should not be regarded as limiting.

### BRIEF DESCRIPTION OF THE DRAWINGS

FIG. 1A shows a schematic block diagram of an illustrative system architecture in accordance with an embodiment.

FIG. 1B shows a schematic block diagram of a general purpose computer in accordance with an illustrative embodiment.

FIG. 2 shows an illustrative floor plan of a mobile trailer for transporting the system to onsite locations for sanitizing wearable equipment.

FIG. 3 shows a left side view of the mobile trailer of FIG. 2.

FIG. 4 shows a right side view of the mobile trailer of FIG. 2.

FIG. 5 shows a front view of the mobile trailer of FIG. 2.

FIG. 6 shows a rear view of the mobile trailer of FIG. 2.

FIG. 7 shows a partial cross-sectional view of the mobile trailer of FIG. 2.

FIG. 8 shows a schematic block diagram of a system in accordance with an illustrative embodiment.

FIG. 9 shows a schematic block diagram of a system in accordance with another illustrative embodiment.

FIGS. 10A and 10B show a schematic block diagram of a loading and unloading system in accordance with an illustrative embodiment.

### DETAILED DESCRIPTION

As noted above, the present invention relates to a system and method for sanitizing wearable equipment.

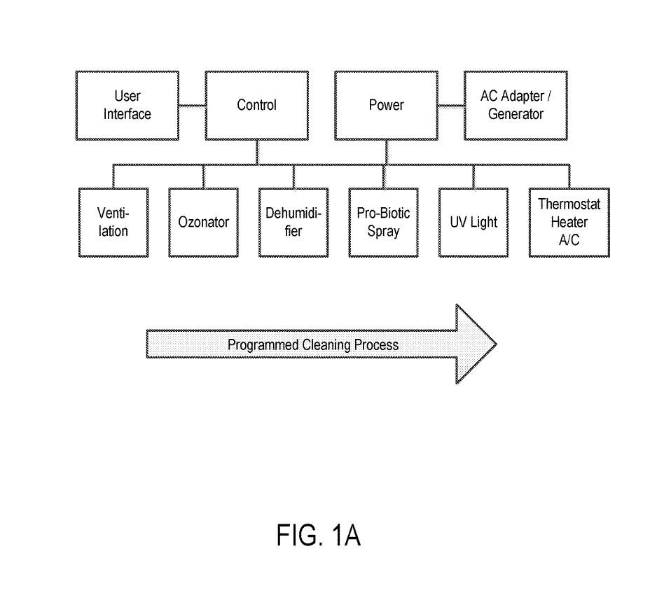

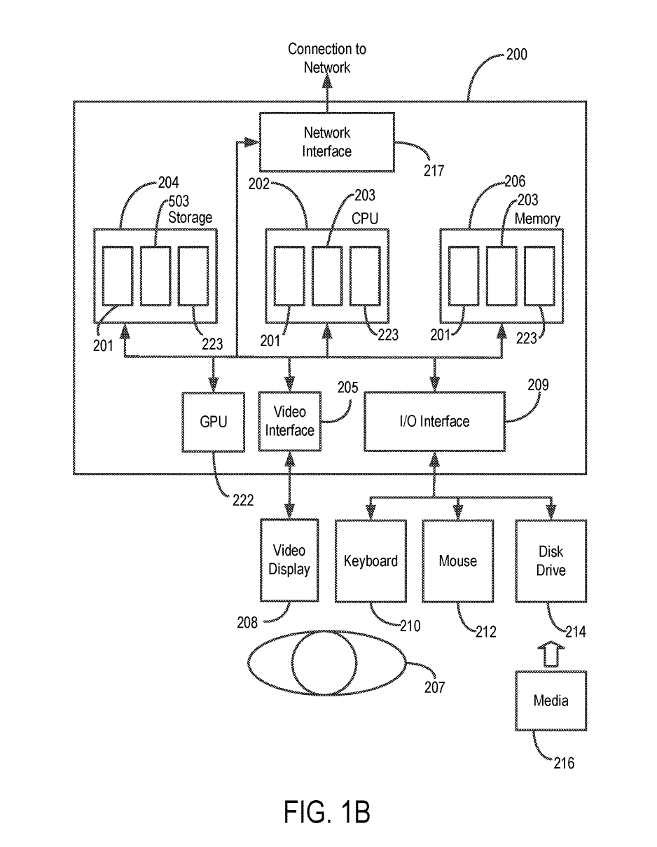

With reference to FIG. 1A, shown is a schematic block diagram of an illustrative system in accordance with an embodiment. In this illustration, the system comprises a control block with a user interface, a power block with an AC or gas generator power source, and various modules that may be controlled by the control module during one or more programmed sanitizing processes. The control module may be for example, a general purpose computer with a processor and a memory for executing computer readable code for executing various instructions. Such a general purpose computer is shown in FIG. 1B, which shows a generic computer device 200 that may include a central processing unit (“CPU”) 202 connected to a storage unit 204 and to a random access memory 506. The CPU 202 may process an operating system 201, application program 203, and data 223. The operating system 201, application program 203, and data 223 may be stored in storage unit 204 and loaded into memory 206, as may be required. Computer device 200 may further include a graphics processing unit (GPU) 222 which is operatively connected to CPU 202 and to memory 206 to offload intensive image processing calculations from CPU 502 and run these calculations in parallel with CPU 202. An operator 207 may interact with the computer device 200 using a video display 208 connected by a video interface 205, and various input/output devices such as a keyboard 210, mouse 212, and disk drive or solid state drive 214 connected by an I/O interface 209. In known manner, the mouse 212 may be configured to control movement of a cursor in the video display 208, and to operate various graphical user interface (GUI) controls appearing in the video display 208 with a mouse button. The disk drive or solid state drive 214 may be configured to accept computer readable media 216. The computer device 200 may form part of a network via a network interface 211, allowing the computer device 200 to communicate with other suitably configured data processing systems (not shown).

Still referring to FIG. 1A, the power block supplies power to the control module, and to each of the modules in the system requiring power. The power may draw AC from an AC power cord, or alternatively the power may be generated by a power generator, such as a gasoline generator for example.

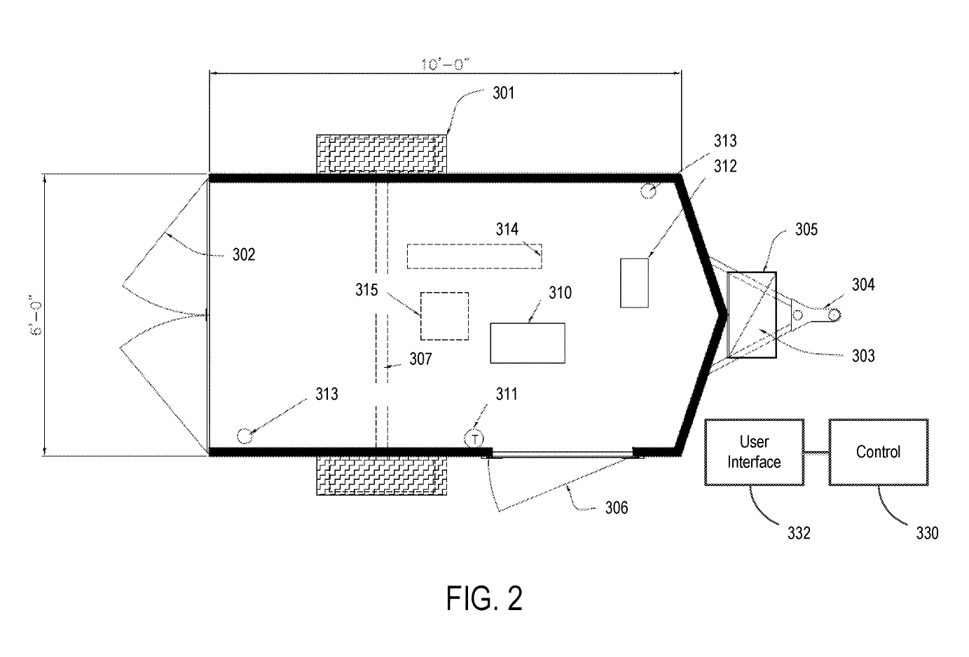

As shown in FIG. 1A, a ventilation module may be provided to control the air flow within the operating environment, which may be a mobile trailer, for example, as illustrated in FIG. 2. A thermostat/heater/air conditioner module may be used to maintain a suitable operating temperature within the mobile trailer, for example during colder winter months when the temperature within the trailer must be kept at a minimum operating temperature for the devices.

Still referring to FIG. 1A, the system includes an ozonator module for applying ozone to wearable equipment being sanitized. The ozonator module emits ozone (O3) to kill organisms/bacteria in equipment, thereby sterilizing and deodorizing the equipment. This step of the process kills the bacteria, hence, should remove any stale smell. This step also reduces the chance of infections if a wearer gets cut and then wears the equipment again.

In another embodiment, the system includes a dehumidifier module, such as a dryer, is used to dehumidify the wearable equipment being cleaned. In an illustrative embodiment, following IICRC (Institute of Inspection Cleaning and Restoration Certification) principles of drying, flowing air is moved around the wearable equipment during the dehumidification process.

Still referring to FIG. 1A, in another embodiment, the system includes a sprayer module to apply a pro-biotic spray to hinder any re-growth of bacteria. The pro-biotic spray is preferably applied from all sides of the wearable equipment being cleaned. Optionally, a perfume may be added into the pro-biotic spray to impart a fresh scent to the wearable equipment while applying the spray.

Still referring to FIG. 1A, in another embodiment, the system includes a UV light source module to subject the wearable equipment to UV light. Germs and bacteria irradiated with a UV light source are killed or inactivated by exposure to short-wavelength UV light. For example, UV light at around 250 nm-260 nm has been found to destroy nucleic acids and disrupt the DNA of germs and microbes. Multiple UV light sources may be positioned around wearable equipment passing through the UV light module in order to irradiate the wearable equipment from multiple angles.

As shown in FIG. 1A, in another embodiment, the system includes a thermostat/heater/air conditioner module which may be controlled to maintain a suitable operating temperature for the ozonator module, the pro-biotic sprayer module, and the UV light source module. The control may be used to control the heater or air conditioner based on the thermostat reading.

Still referring to FIG. 1A, in another embodiment, the system includes ventilation for ventilating the operating environment. The ventilation may comprise one or more fans controlled by the control to circulate ambient air within the operating environment by taking in fresh air, and venting out stale air. The ventilation and the thermostat/heater/air conditioner may be controlled together by the control in order to maintain temperature and humidity within acceptable operating ranges.

Now referring to FIG. 2, shown is an illustrative floor plan of a mobile trailer for transporting the system to onsite locations for sanitizing wearable equipment. The identified parts of the mobile trailer are a wheel fender 301, doors 302, power generator 303, trailer hitch 304, a generator tray 305, and side door 306. A trailer axle 307 is shown in broken lines.

Inside the mobile trailer, the identified parts are an ozonator 310, thermostat 311, dehumidifier 312, and automatic probiotic spray 313, which may be provided at multiple locations. An air treatment unit 314 may be located at a ceiling vent. A roof skylight and vent 315 is also provided.

In an embodiment, a control module 330 with a user interface 332 may be provided externally to the mobile trailer, such that an operator may control one or more sanitizing modules, regulate humidity and temperature, and monitor the sanitizing process. The control module with a user interface may be wired to one or more modules inside the mobile trailer, or may be connected wirelessly to communicate bi-directionally with one or more modules.

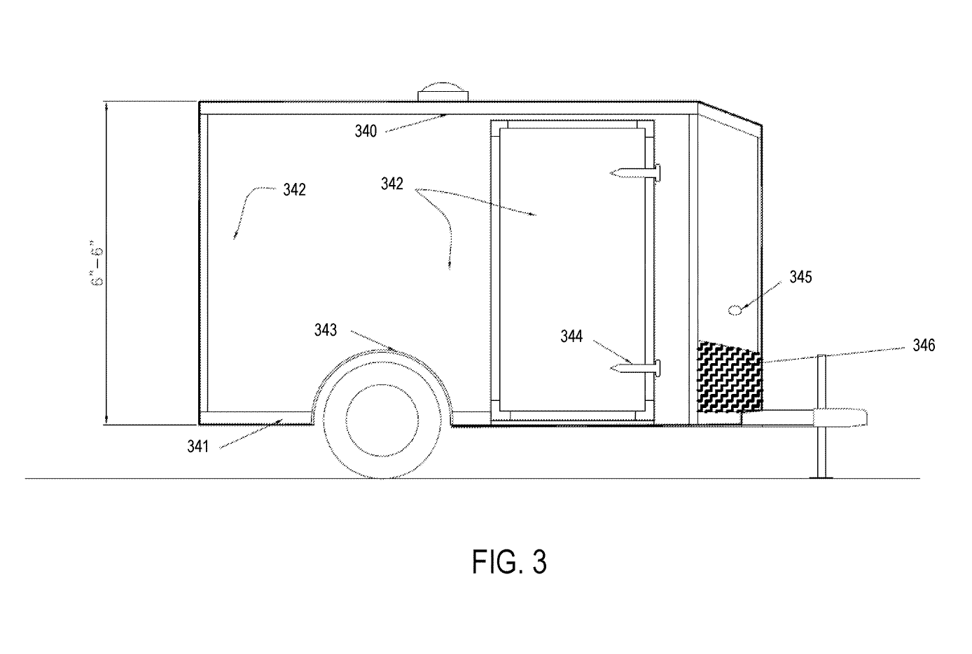

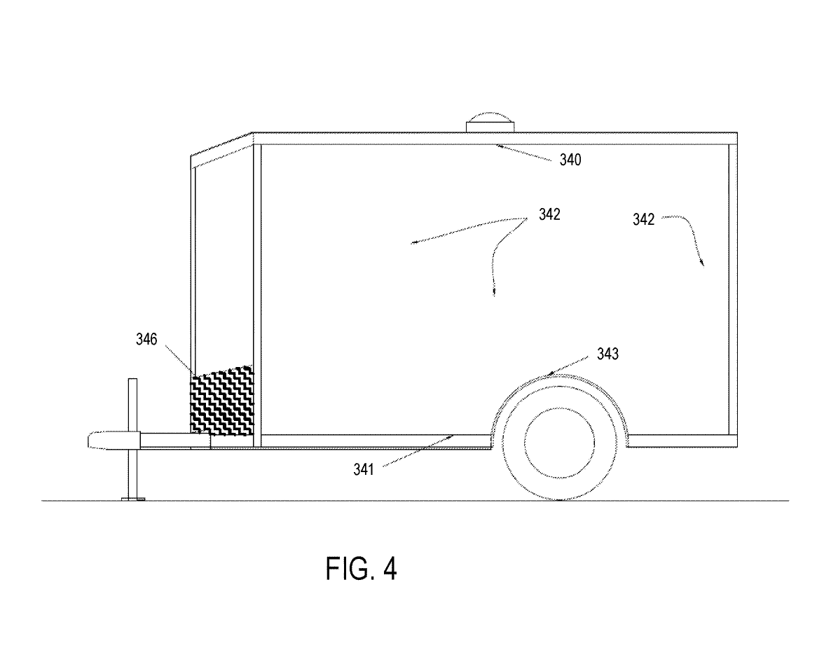

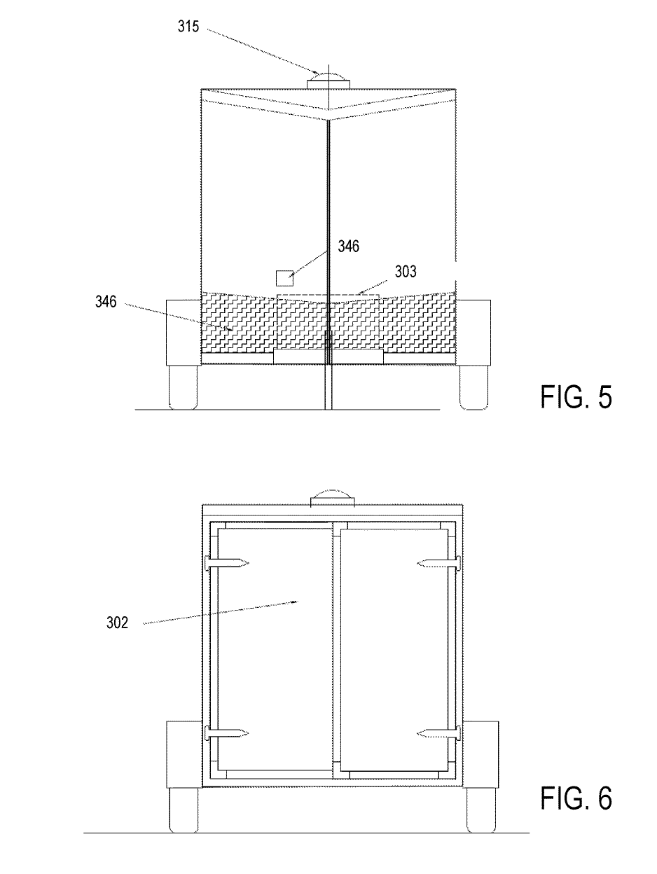

FIGS. 3 to 6 show various views of the mobile trailer of FIG. 2 from different points of view. Shown in FIGS. 3 and 4 are the following identified parts including a top trim 340, bottom trim 341, seamless aluminum siding 342, plate fender 343, door hinges 344, electrical cord access 345, and a protective guard plate 346. FIG. 5 and FIG. 6 show some of the features from a different point of view.

Still referring to FIGS. 3 to 6, in an embodiment, the mobile trailer shows one possible configuration for the various modules, where the whole trailer is used as an operating environment for sanitizing the wearable equipment. In this example, a power generator is placed outside of the mobile trailer cabin, and near the hitch.

A dehumidifier is placed inside the mobile trailer cabin to regulate humidity. A thermostat is provided to regulate temperature within the mobile trailer cabin using a heater and/or air conditioner depending on the season and operating climate.

Automatic probiotic sprays are positioned at opposite corners of the trailer, and are configured to spray the entire trailer cabin when the cabin is sealed for the sanitizing operation. This seal may be provided by way of appropriate seals provided on the

In an embodiment, one or more UV lights may be positioned to irradiate wearable equipment placed in a sanitizing area. The UV lights are positioned to expose the wearable equipment from multiple angles for best coverage. Optionally, the wearable equipment may be mounted on a rotating table or cage to expose the wearable equipment from different angles.

In an embodiment, an ozonator is positioned in the mobile trailer cabin to release ozone into the entire trailer cabin when the cabin is sealed for the sanitizing operation.

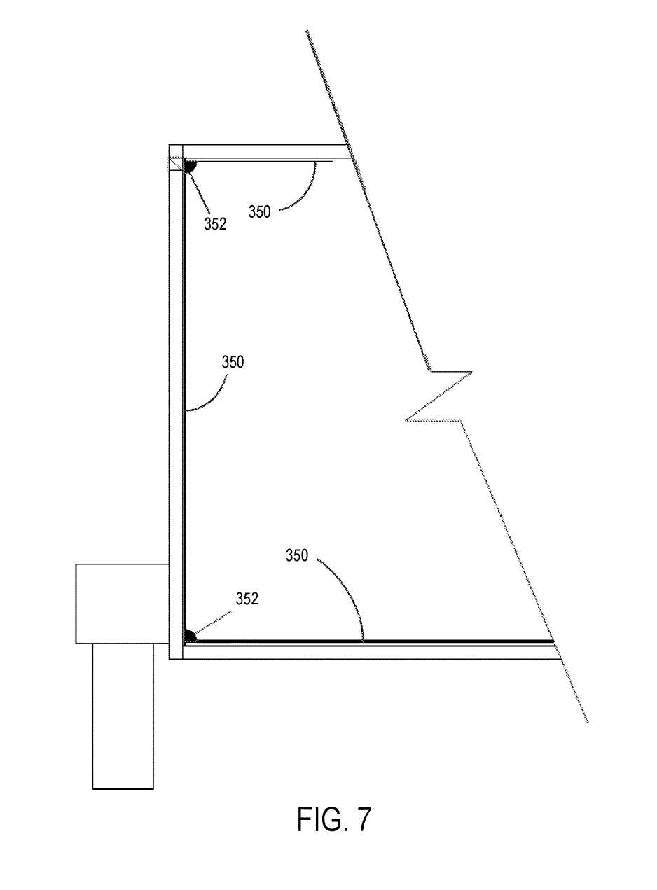

FIG. 7 shows a partial cross-sectional view of the mobile trailer of FIG. 2. In an illustrative embodiment, the mobile trailer unit may be suitably assembled from wood and aluminum panels 350. The walls may be provided with various hanging brackets, hooks and bags for equipment positioning. On the floor of the unit, interlocking rubber tile flooring may be used to protect the floor from liquid spills, and to prevent slippage when operators are inside the cabin. Caulking 352 may be used at any internal corners or wall panel joins to ensure sealing of any cracks.

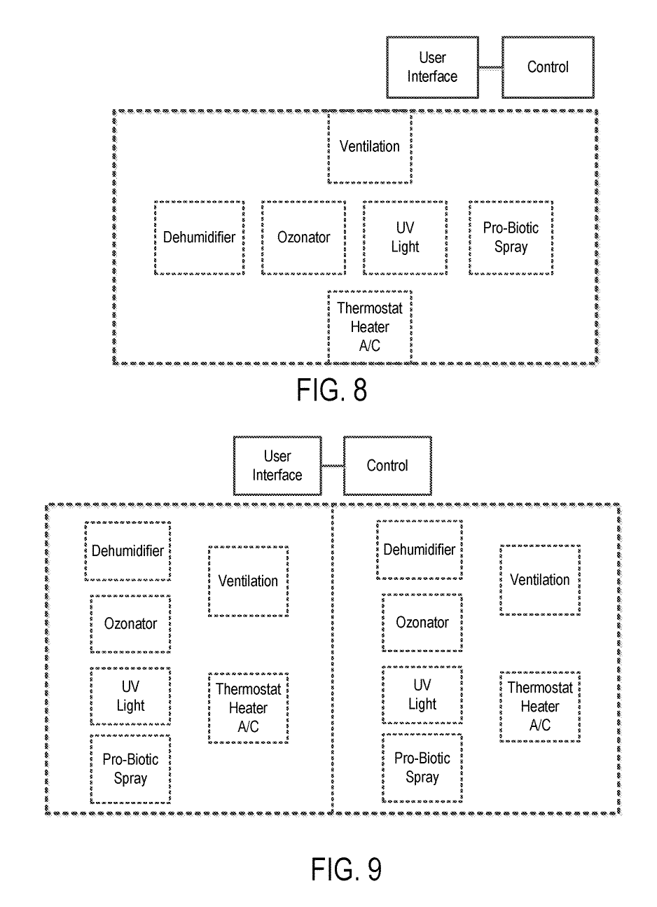

FIG. 8 shows a schematic block diagram of a stationary configuration of the system in accordance with an illustrative embodiment. This embodiment is suitable where the wearable equipment to be cleaned is substantially of the same type, and the entire cabin can be used to hold the wearable equipment to be cleaned and each of the different types of sanitizing modules as described above. A ventilator, and a thermostat/heater/air conditioner may be controlled to maintain a suitable humidity and operating temperature for the entire compartment or cabin, during a sanitizing operation. The modules can be controlled via wired or wireless control from outside the sealed cabin by an operator.

FIG. 9 shows a schematic flow chart of a system in accordance with another illustrative embodiment. In this case, the cabin is divided into multiple compartments, where each compartment includes the different types of sanitizing modules as described above. In operation, different types of wearable equipment to be cleaned may be sorted and placed into different compartments, and be subjected to a different sanitizing program in each compartment. The compartments may be operated simultaneously, and if each compartment is undergoing a different sanitizing program, one compartment can be opened to remove sanitized wearable equipment, and replaced with another load of wearable equipment to be cleaned. A ventilator, and a thermostat/heater/air conditioner may be controlled to maintain a suitable humidity and operating temperature in each compartment, during a sanitizing operation.

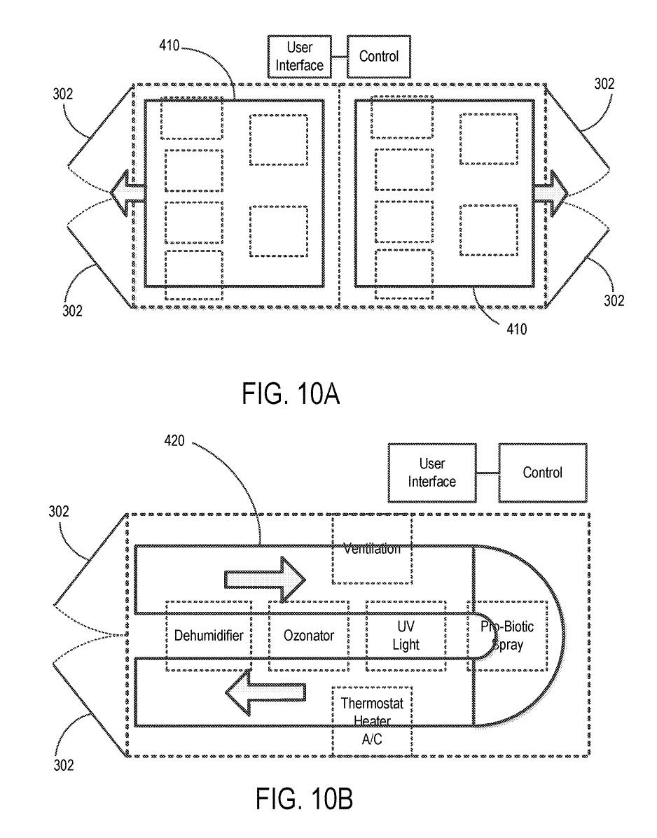

Now referring to FIGS. 10A and 10B, shown are schematic block diagrams of a loading and unloading system. In the example shown in FIG. 10A, a rack or a straight line conveyer 410 allows loading and unloading at one end of the sealable enclosure. The rack or conveyer 410 may be adapted to be positioned below or above any cleaning modules inside the mobile cleaning unit to provide a large area for loading wearable equipment to be cleaned. The rack or conveyor may be operated to allow wearable equipment to be loaded and unloaded.

Alternatively, as shown in FIG. 10B, a conveyor layout allows the wearable equipment to be loaded from one side, and unloaded from another side via sealable access doors 302.

This, in an aspect, there is provided a system for sanitizing wearable equipment, comprising: a plurality of sanitizing modules arranged on a platform within a sealable enclosure; and a control module with a user interface for remotely controlling the plurality of sanitizing modules.

In an embodiment, the platform is mobile.

In another embodiment, the platform is a mobile trailer having a mobile power generator.

In another embodiment, the plurality of sanitizing modules comprises one or more of an ozonator module, a pro-biotic spray module and a UV light module.

In another embodiment, the system further comprises a dehumidifier module and a thermostat module for controlling one or more of a heater and an air conditioner.

In another embodiment, the control is configured to control one or more automated sanitizing programs based on the type of wearable equipment to be sanitized.

In another embodiment, the sealable enclosure is accessible via a sealable access door.

In another embodiment, the sealable access door provides access to a loading area for loading wearable equipment to be sanitized.

In another embodiment, the system further comprises a rack for loading and unloading wearable equipment to be cleaned without having to enter the sealable enclosure.

In another embodiment, the system further comprises a conveyor for loading and unloading wearable equipment to be cleaned without having to enter the sealable enclosure.

In another aspect, there is provided a method of sanitizing wearable equipment, comprising: providing a plurality of sanitizing modules arranged on a platform within a sealable enclosure; and providing a control module with a user interface for remotely controlling the plurality of sanitizing modules.

In another embodiment, the platform is mobile.

In another embodiment, the platform is a mobile trailer having a mobile power generator.

In another embodiment, the method further comprises sanitizing the wearable equipment by utilizing one or more of an ozonator module, a pro-biotic spray module and a UV light module.

In another embodiment, the method further comprises utilizing a dehumidifier module and a thermostat module for controlling one or more of a heater and an air conditioner.

In another embodiment, the control is configured to control one or more automated sanitizing programs based on the type of wearable equipment to be sanitized.

In another embodiment, the method further comprises providing access to the sealable enclosure via a sealable access door.

In another embodiment, the method further comprises providing a loading area adjacent the sealable access door for loading wearable equipment to be sanitized.

In another embodiment, the method further comprises providing a rack for loading and unloading wearable equipment to be cleaned without having to enter the sealable enclosure.

In another embodiment, the method further comprises providing a conveyor for loading and unloading wearable equipment to be cleaned without having to enter the sealable enclosure. While various illustrative embodiments have been described above by way of example, it will be appreciated that various changes and modifications may be made without departing from the scope of the invention, which is defined by the following claims

1. A system for sanitizing wearable equipment, comprising:
a plurality of sanitizing modules arranged on a platform within a sealable enclosure; and
a control module with a user interface for remotely controlling the plurality of sanitizing modules.

2. The system of claim 1, wherein the platform is mobile.

3. The system of claim 2, wherein the platform is a mobile trailer having a mobile power generator.

4. The system of claim 1, wherein the plurality of sanitizing modules comprises one or more of an ozonator module, a pro-biotic spray module and a UV light module.

5. The system of claim 1, further comprising a dehumidifier module and a thermostat module for controlling one or more of a heater and an air conditioner.

6. The system of claim 1, wherein the control is configured to control one or more automated sanitizing programs based on the type of wearable equipment to be sanitized.

7. The system of claim 1, wherein the sealable enclosure is accessible via a sealable access door.

8. The system of claim 7, wherein the sealable access door provides access to a loading area for loading wearable equipment to be sanitized.

9. The system of claim 8, further comprising a rack for loading and unloading wearable equipment to be cleaned without having to enter the sealable enclosure.

10. The system of claim 8, further comprising a conveyor for loading and unloading wearable equipment to be cleaned without having to enter the sealable enclosure.

11. A method of sanitizing wearable equipment, comprising:
providing a plurality of sanitizing modules arranged on a platform within a sealable enclosure; and
providing a control module with a user interface for remotely controlling the plurality of sanitizing modules.

12. The method of claim 11, wherein the platform is mobile.

13. The method of claim 12, wherein the platform is a mobile trailer having a mobile power generator.

14. The method of claim 11, further comprising sanitizing the wearable equipment by utilizing one or more of an ozonator module, a pro-biotic spray module and a UV light module.

15. The method of claim 11, further comprising utilizing a dehumidifier module and a thermostat module for controlling one or more of a heater and an air conditioner.

16. The method of claim 11, wherein the control is configured to control one or more automated sanitizing programs based on the type of wearable equipment to be sanitized.

17. The method of claim 1, further comprising providing access to the sealable enclosure via a sealable access door.

18. The method of claim 17, further comprising providing a loading area adjacent the sealable access door for loading wearable equipment to be sanitized.

19. The method of claim 18, further comprising providing a rack for loading and unloading wearable equipment to be cleaned without having to enter the sealable enclosure.

20. The method of claim 18, further comprising providing a conveyor for loading and unloading wearable equipment to be cleaned without having to enter the sealable enclosure.
